(19) United States
(12) Patent Application Publication
(10) Pub. No.: US 2005/0201969 A1
(43) Pub. Date: Sep. 15, 2005

(54) Stable cosmetics

(75) Inventors: Albrecht Dorschner, Hamburg (DE); Stephan Ruppert, Hamburg (DE); Ursula Jensen, Hamburg (DE); Elke Willems, Hamburg (DE); Christian Frese, Hamburg (DE); Martin Kauffeldt, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/064,213

(22) Filed: Feb. 23, 2005

(30) Foreign Application Priority Data
Feb. 23, 2004 (DE) 10 2004 009 149.8

(51) Int. Cl. A61K 7/06; A61K 7/11

(52) U.S. Cl. 424/70.13

(57) ABSTRACT

A cosmetic preparation comprising one or more labile ingredients, and one or more pregelatinized, crosslinked starch derivatives.

### FIELD OF THE INVENTION

The present invention relates to a cosmetic preparation comprising one or more labile ingredients and one or more pregelatinized, crosslinked starch derivatives.

### BACKGROUND OF THE INVENTION

The desire for clean skin is probably as old as mankind since dirt, perspiration and residues of dead skin particles offer ideal breeding grounds for pathogens and parasites of all types. The desire for body hygiene was steadily intensified when, in the 1960s, besides the “classical” soap, it was also possible to formulate liquid detergents with newly developed synthetic surfactants. Since this time, daily life without bathing and showering has become inconceivable. Nowadays, a large number of products for cleansing the various parts of the body are available to consumers.

A particular group of skin cleansing products here are facial cleansing products. Since the facial skin is particularly sensitive, especially mild products which do not irritate the skin are used for facial cleansing. In most cases, gels, i.e., semisolid, more or less transparent systems are used here.

Cleansing gels comprise water, surfactants and thickener (gel former) as main constituents.

The surfactants are the washing-active substances in the cleansing gels. As result of their specific molecular structure with in each case one hydrophilic (water-attracting) and hydrophobic (water-repelling) group in the same molecule, they serve to reduce the surface tension of water, as result of which soil removal becomes easier. A distinction is made between anionic, cationic, amphoteric and non-ionic surfactants depending on the charge state. On account of their ability to reduce the surface tension of water, surfactants bring about foaming of the preparation.

In the cleansing gel, the thickeners, also called gel formers, form a three-dimensional network in which the liquid (usually water) is immobilized. The thickeners used are in most cases polymers such as polyacrylates. These are added to the preparation to be thickened at neutral pH and then deprotonated by adding bases, as a result of which the preparation converts to a viscous gel.

Moreover, cleansing gels can comprise a number of further ingredients, for example perfume substances or cosmetic active ingredients and care substances.

However, conventional cleansing gels prepared in accordance with the prior art have the disadvantage that they are unable to comprise labile ingredients, particularly if they are stored in transparent (i.e. clearly see-through) or translucent (i.e. milky see-through) packagings. However, transparent packagings in particular are a packaging form of particular preference for gels since by virtue of this type of packaging the colour and/or effect substances advantageously present in the gels can be visually completely appreciated. In accordance with the prior art, this problem could at best be solved by adding UV photoprotective filters to the preparation or to the packaging material (as a result of which, inter alia, the production costs of the cosmetics increase) or by omitting (photo) labile ingredients.

It was therefore the object of the present invention to overcome the shortcomings of the prior art.

### SUMMARY OF THE INVENTION

Surprisingly, the object has now been achieved by a cosmetic preparation comprising one or more labile ingredients, and one or more pregelatinized, crosslinked starch derivatives.

In addition, the object has surprisingly been achieved by a cosmetic which is formed from

a) a transparent or translucent packaging and

b) a cosmetic preparation comprising one or more labile ingredients, and one or more pregelatinized, crosslinked starch derivatives.

Furthermore, the object was achieved, in a manner which was not obvious to the person skilled in the art, through the use of pregelatinized, crosslinked starch derivatives for stabilizing labile ingredients in cosmetics.

The invention was therefore particularly surprising since pregelatinized, crosslinked starch derivatives themselves have no noteworthy UV photoprotective filter properties.

Although the prior art in WO 98/01109 describes the increase in the odour stability and storage stability due to starch derivatives, this specification was unable to point the way to the present invention since the effects described in WO 98/01109 concern an increase in the thermal stability and an increase in the microbial stability. However, as is known to the chemist, thermal and photochemical reactions are based on completely different reaction mechanisms meaning that a thermal stability of a substance cannot be used to deduce its photostability (and vice versa).

The disclosures of DE 102 16 504, WO 96/22073 and U.S. Pat. No. 6,248,338 were also unable to point the way to the present invention.

### DETAILED DESCRIPTION OF THE INVENTION

According to the invention, “stability” is understood in particular as meaning the colour stability of the preparations. The preparations and uses according to the invention lead in particular to the preparations no longer discolouring under the effect of light.

According to the invention, labile ingredients are understood as meaning compounds which discolour over the course of time and thus lead to a discoloration (colour change) of the preparation comprising them. In particular, labile ingredients are understood as meaning compounds which discolour under the influence of light (i.e. are photolabile) and thus lead to a discoloration (colour change) of the preparation comprising them.

It is advantageous according to the invention if the pregelatinized, crosslinked starch derivatives used are hydroxypropylated phosphate esters. Of particular advantage are those starch derivatives as described in U.S. Pat. No. 6,248,338, particularly advantageously hydroxypropyldistarch phosphate. Very particular preference here is given to the use of a hydroxypropyldistarch phosphate as is sold as the product Structure® XL by National Starch.

Preparations, cosmetics or uses advantageous according to the invention are characterized in that the pregelatinized, crosslinked starch derivatives are present therein in a total concentration of from 0.05 to 5% by weight and preferably in a total concentration of from 0.1 to 2% by weight, in each case based on the total weight of the preparation.

In one embodiment preferred according to the invention, preparations, cosmetics or uses advantageous according to the invention are characterized in that the labile ingredients used are licochalcone A and/or ubiquinone Q-10.

Here, it is advantageous according to the invention if the total amount of licochalcone A and ubiquinone Q-10 in the preparation is from 0.001 to 0.3% by weight, and preferred according to the invention if the total amount of licochalcone A and ubiquinone Q-10 in the preparation is from 0.002 to 0.2% by weight, based on the total weight of the preparation.

According to the invention, the concentration of licochalcone A in the preparation is advantageously from 0.001 to 0.1% by weight and preferably from 0.002 to 0.05% by weight, in each case based on the total weight of the preparation.

Licochalcone A is an effective constituent of the aqueous extract from Glycyrrhiza inflata.

The species Glycyrrhiza inflata belongs, like the liquorice which is officinal in Europe Glycyrrhiza glabra, to the genus Glycyrrhiza, which belongs to the Fabaceae plant family (pea plants). The drug Radix Glycyrrhizae inflatae, i.e. the root of the plant, is customary, for example, in Far Eastern medicine.

One constituent of the aqueous extract from Radix Glycyrrhizae inflatae is licochalcone A. For the purposes of the present invention, it is therefore advantageous to add the licochalcone A according to the invention in the form of the aqueous extract from Radix Glycyrrhizae inflatae to the preparation according to the invention.

According to the invention, the concentration of ubiquinone Q-10 in the preparation is advantageously from 0.001 to 0.1% by weight and preferably from 0.002 to 0.05% by weight, in each case based on the total weight of the preparation.

In a further embodiment preferred according to the invention, preparations, cosmetics or uses advantageous according to the invention are characterized in that the labile ingredients are perfume substances.

Here, it is advantageous according to the invention if the total amount of perfume substances in the preparation is from 0 to 2.0% by weight, and preferred according to the invention if the total amount of perfume substances in the preparation is from 0 to 0.8% by weight, in each case based on the total weight of the preparation.

Mixtures of licochalcone A, ubiquinone Q-10 and perfume substances can also be used according to the invention advantageously in all mixing ratios (two or three components) in the preparation or use according to the invention.

The preparations according to the invention can, moreover, comprise a large number of further cosmetic active ingredients, auxiliaries and additives.

According to the invention, the preparation according to the invention or the preparation in which the use according to the invention is realized is advantageously a water-based preparation. According to the invention, a water concentration of from 60 to 90% by weight is advantageous, preferably from 70 to 90% by weight and very particularly preferably from 75 to 85% by weight, in each case based on the total weight of the preparation.

According to the invention, the preparation according to the invention or the preparation in which the use according to the invention is realized is a surfactant-containing preparation.

Surfactants are generally classified according to the type and charge of the hydrophilic molecular moiety. Four groups can be differentiated here:

anionic surfactants,

cationic surfactants,

amphoteric surfactants and

nonionic surfactants.

The following surfactants can be used advantageously according to the invention:

A. Anionic Surfactants

Anionic surfactants to be used advantageously are acylamino acids (and salts thereof), such as

1. acyl glutamates, for example sodium acyl glutamate, di-TEA-palmitoyl aspartate and sodium caprylic/capric glutamate,
2. acylpeptides, for example palmitoyl-hydrolysed milk protein, sodium cocoyl-hydrolysed soya protein and sodium/potassium cocoyl-hydrolysed collagen,
3. sarcosinates, for example myristoyl sarcosinate, TEA-lauroyl sarcosinate, sodium lauroyl sarcosinate and sodium cocoyl sarcosinate,
4. taurates, for example sodium lauroyl taurate and sodium methyl cocoyl taurate,
5. acyl lactylates, lauroyl lactylate, caproyl lactylate
6. alaninates
   carboxylic acids and derivatives, such as
1. carboxylic acids, for example lauric acid, aluminium stearate, magnesium alkanolate and zinc undecylenate,
2. ester carboxylic acids, for example calcium stearoyl lactylate, laureth-6 citrate and sodium PEG-4 lauramide carboxylate,
3. ether carboxylic acids, for example sodiumlaureth-13 carboxylate and sodium PEG-6 cocamide carboxylate,
   phosphoric esters and salts, such as, for example, DEA-oleth-10 phosphate and dilaureth-4 phosphate,
   sulphonic acids and salts, such as
1. acyl isethionates, e.g. sodium/ammonium cocoyl isethionate,
2. alkylarylsulphonates,
3. alkylsulphonates, for example sodium cocomonoglyceride sulphate, sodium C₁₂₋₁₄ olefinsulphonate, sodium lauryl sulphoacetate and magnesium PEG-3 cocamide sulphate,
4. sulphosuccinates, for example dioctyl sodium sulphosuccinate, disodium laureth sulphosuccinate, disodium lauryl sulphosuccinate, disodium undecylenamido-MEA sulphosuccinate and PEG-5 lauryl citrate sulphosuccinate.
   and
   sulphuric esters, such as
1. alkyl ether sulphates, for example sodium, ammonium, magnesium, MIPA, TIPA laureth sulphate, sodium myreth sulphate and sodium C₁₂₋₁₃-pareth sulphate,
2. alkyl sulphates, for example sodium, ammonium and TEA lauryl sulphate.
   B. Cationic Surfactants
   Cationic surfactants to be used advantageously are
1. alkylamines,
2. alkylimidazoles,
3. ethoxylated amines and
4. quaternary surfactants
5. ester quats

Quaternary surfactants contain at least one N atom which is covalently bonded to 4 alkyl and/or aryl groups. Irrespective of the pH, this leads to a positive charge. Advantageous quaternary surfactants are alkylbetaine, alkylamidopropylbetaine and alkylamidopropylhydroxysultaine. For the purposes of the present invention, cationic surfactants can also preferably be chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulphates, alkylpyridinium salts, for example lauryl- or cetylpyridinium chloride, imidazoline derivatives and compounds with cationic character, such as amine oxides, for example alkyldimethylamine oxides or alkylaminoethyldimethylamine oxides. Cetyltrimethylammonium salts in particular are to be used advantageously.

C. Amphoteric Surfactants

Amphoteric surfactants to be used advantageously are

1. acyl/dialkylethylenediamine, for example sodium acyl amphoacetate, disodium acyl amphodipropionate, disodium alkylamphodiacetate, sodium acylamphohydroxypropylsulphonate, disodium acyl amphodiacetate and sodium acyl amphopropionate,
2. N-alkylamino acids, for example aminopropylalkylglutamide, alkylaminopropionic acid, sodium alkylimidodipropionate and lauroamphocarboxyglycinate.
   D. Nonionic Surfactants
   Nonionic surfactants to be used advantageously are
1. alcohols,
2. alkanolamides, such as cocamide MEA/DEA/MIPA,
3. amine oxides, such as cocoamidopropylamine oxide,
4. esters which are produced by esterification of carboxylic acids with ethylene oxide, glycerol, sorbitan or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside,
6. sucrose esters, sucrose ethers
7. polyglycerol esters, diglycerol esters, monoglycerol esters
8. methylglucose esters, esters of hydroxyl acids

Also advantageous is the use of a combination of anionic and/or amphoteric surfactants with one or more nonionic surfactants.

According to the invention, the total concentration of surfactants in the preparations according to the invention is advantageously from 0.5 to 15% by weight and according to the invention is preferably from 2 to 10% by weight, in each case based on the total weight of the preparation.

The surfactant mixtures particularly preferred according to the invention and their particularly preferred use concentrations are disclosed in the example formulations.

Preparations according to the invention can also advantageously comprise thickeners. Suitable thickeners are homopolymers of acrylic acid with a molecular weight of from 2 000 000 to 6 000 000, such as, for example, commercial product Carbopols. Further thickeners are sold under the names Carbopol 940, Carbopol EDTA 2001 or Modarez V 600 PX. Also suitable are polymers of acrylic acid and acrylamide (sodium salt) with a molecular weight of from 2 000 000 to 6 000 000, such as, for example, Hostacerin PN 73 or the sclerotium gum sold under the name Amigel. Also suitable are copolymers of acrylic acid or of methacrylic acid, such as, for example Carbopol 1342 or Pemulen TRI. Further types of thickeners are polyglycols, cellulose derivatives, in particular hydroxyalkylcelluloses, and alignates, carrageenan and inorganic thickeners, such as, for example, natural or synthetic bentonites.

According to the invention, the total concentration of thickeners in the preparations according to the invention is advantageously from 0.01 to 2% by weight and according to the invention preferably from 0.1 to 1% by weight, in each case based on the total weight of the preparation.

The thickeners particularly preferred according to the invention and their particularly preferred use concentrations are disclosed in the example formulations.

The preparation according to the invention advantageously comprises one or more conditioners. Conditioners preferred according to the invention are, for example, all compounds which are listed in the International Cosmetic Ingredient Dictionary and Handbook (Volume 4, editor: R. C. Pepe, J. A. Wenninger, G. N. McEwen, The Cosmetic, Toiletry, and Fragrance Association, 9^{th} edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all of the compounds listed in EP 0934956 (pp. 11-13) under water soluble conditioning agent and oil soluble conditioning agent. Further conditioners advantageous according to the invention are, for example, the compounds named according to the International Nomenclature for Cosmetic Ingredients (INCI) as polyquaternium. Thus, for example, polyquaternium-1 to polyquaternium-56, but also the polyethylene glycols and polypropylene glycols are conditioners advantageous according to the invention.

The preparation according to the invention can advantageously comprise abrasives with an average particle size of less than 400 μm, in an amount of from 0.1 to 3% by weight, in each case based on the total weight of the preparation, the abrasives preferably consisting of polyethylene.

The cosmetic preparations according to the invention can comprise a number of pigments. The dyes and colour pigments can be chosen from the corresponding positive list of the Cosmetics Directive or the EC list of cosmetic colorants. In most cases, they are identical to the dyes approved for foods. Advantageous colour pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet. It is particularly advantageous to choose the dyes and/or colour pigments from the following list. The Colour Index Numbers (CIN) are given in the Rowe Colour Index, 3^{rd} edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-Dinitrohydroxynaphthalene-7-sulphonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-Chloro-4′-nitro-1′-phenylazo)-2- | 12085 | Red |
| hydroxynaphthalene | | |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′-sulphodiethylamido-1′-phenylazo)-3- | 12490 | Red |
| hydroxy-5″-chloro-2″,4″-dimethoxy-2-naphthanilide | | |
| Disperse Yellow 16 | 12700 | Yellow |
| 1-(4-Sulpho-1-phenylazo)-4-aminobenzene-5- | 13015 | Yellow |
| sulphonic acid | | |
| 2,4-Dihydroxyazobenzene-4′-sulphonic acid | 14270 | Orange |
| 2-(2,4-Dimethylphenylazo-5-sulpho)-1- | 14700 | Red |
| hydroxynaphthalene-4-sulphonic acid | | |
| 2-(4-Sulpho-1-naphthylazo)-1-naphthol-4- | 14720 | Red |
| sulphonic acid | | |
| 2-(6-Sulpho-2,4-xylylazo)-1-naphthol-5-sulphonic | 14815 | Red |
| acid | | |
| 1-(4′-Sulphophenylazo)-2-hydroxynaphthalene | 15510 | Orange |
| 1-(2-Sulpho-4-chloro-5-carboxy-1-phenylazo)-2- | 15525 | Red |
| hydroxy-naphthalene | | |
| 1-(3-Methylphenylazo-4-sulpho)-2- | 15580 | Red |
| hydroxynaphthalene | | |
| 1-(4′,(8′)-Sulphonaphthylazo)-2- | 15620 | Red |
| hydroxynaphthalene | | |
| 2-Hydroxy-1,2′-azonaphthalene-1′-sulphonic acid | 15630 | Red |
| 3-Hydroxy-4-phenylazo-2-naphthylcarboxylic acid | 15800 | Red |
| 1-(2-Sulpho-4-methyl-1-phenylazo)-2- | 15850 | Red |
| naphthylcarboxylic acid | | |
| 1-(2-Sulpho-4-methyl-5-chloro-1-phenylazo)-2- | 15865 | Red |
| hydroxy-naphthalene-3-carboxylic acid | | |
| 1-(2-Sulpho-1-naphthylazo)-2-hydroxynaphthalene-3- | 15880 | Red |
| carboxylic acid | | |
| 1-(3-Sulpho-1-phenylazo)-2-naphthol-6-sulphonic acid | 15980 | Orange |
| 1-(4-Sulpho-1-phenylazo)-2-naphthol-6-sulphonic acid | 15985 | Yellow |
| Allura Red | 16035 | Red |
| 1-(4-Sulpho-1-naphthylazo)-2-naphthol-3,6- | 16185 | Red |
| disulphonic acid | | |
| Acid Orange 10 | 16230 | Orange |
| 1-(4-Sulpho-1-naphthylazo)-2-naphthol-6,8- | 16255 | Red |
| disulphonic acid | | |
| 1-(4-Sulpho-1-naphthylazo)-2-naphthol-3,6,8- | 16290 | Red |
| trisulphonic acid | | |
| 8-Amino-2-phenylazo-1-naphthol-3,6-disulphonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulpho-1-phenylazo)-1-(4-sulphophenyl)-5- | 19140 | Yellow |
| hydroxy-pyrazolone-3-carboxylic acid | | |
| Pigment Yellow 16 | 20040 | Yellow |
| 2,6-(4′-Sulpho-2″,4″-dimethyl)bisphenylazo)1,3- | 20170 | Orange |
| dihydroxybenzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulpho-1″-phenylazo)-7′-sulpho-1′- | 27755 | Black |
| naphthylazo]-1-hydroxy-7-aminonaphthalene-3,6- | | |
| disulphonic acid | | |
| 4′-[(4″-Sulpho-1″-phenylazo)-7′-sulfo-1′-naphthylazo]- | 28440 | Black |
| 1-hydroxy-8-acetylaminonaphthalene-3,5-disulphonic | | |
| acid | | |
| Direct Orange 34, 39, 44, 46, 60 | 40215 | Orange |
| Food Yellow | 40800 | Orange |
| trans-β-apo-8′-carotenaldehyde (C₃₀) | 40820 | Orange |
| trans-apo-8′-carotenic acid (C₃₀)-ethyl ester | 40825 | Orange |
| Canthaxanthin | 40850 | Orange |
| Acid Blue 1 | 42045 | Blue |
| 2,4-Disulpho-5-hydroxy-4′-4″- | 42051 | Blue |
| bis(diethylamino)triphenyl-carbinol | | |
| 4-[(-4-N-Ethyl-p-sulphobenzylamino)phenyl(4- | 42053 | Green |
| hydroxy-2-sulphophenyl)(methylene)-1-(N-ethyl-N- | | |
| p-sulphobenzyl)-2,5-cyclohexadienimine] | | |
| Acid Blue 7 | 42080 | Blue |
| (N-Ethyl-p-sulphobenzylamino)phenyl(2- | 42090 | Blue |
| sulphophenyl)-methylene(N-ethyl-N-p-sulphobenzyl)- | | |
| Δ^{2,5}-cyclohexadienimine | | |
| Acid Green 9 | 42100 | Green |
| Diethyldisulphobenzyl-di-4-amino-2-chlorodi-2- | 42170 | Green |
| methyl-fuchsonimmonium | | |
| Basic Violet 14 | 42510 | Violet |
| Basic Violet 2 | 42520 | Violet |
| 2′-Methyl-4′-(N-ethyl-N-m-sulphobenzyl)amino-4″-(N- | 42735 | Blue |
| diethyl)amino-2-methyl-N-ethyl-N-m- | | |
| sulphobenzylfuchsonimmonium | | |
| 4′-(N-Dimethyl)amino-4″-(N-phenyl)aminonaphtho-N- | 44045 | Blue |
| dimethylfuchsonimmonium | | |
| 2-Hydroxy-3,6-disulpho-4,4′- | 44090 | Green |
| bisdimethylaminonaphtho-fuchsonimmonium | | |
| Acid Red 52 | 45100 | Red |
| 3-(2′-Methylphenylamino)-6-(2′-methyl-4′- | 45190 | Violet |
| sulphophenylamino)-9-(2″-carboxyphenyl) xanthenium | | |
| salt | | |
| Acid Red 50 | 45220 | Red |
| Phenyl-2-oxyfluorone-2-carboxylic acid | 45350 | Yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-Diiodofluorescein | 45425 | Red |
| 2,4,5,7-Tetraiodofluorescein | 45430 | Red |
| Quinophthalone | 47000 | Yellow |
| Quinophthalonedisulphonic acid | 47005 | Yellow |

It may also be favourable to choose as dye one or more substances from the following group: 2,4-dihydroxyazobenzene, 1-(2′-chloro-4′-nitro-1′-phenylazo)-2-hydroxynaphthalene, Ceres Red, 2-(4-sulpho-1-naphthylazo)-1-naphthol-4-sulphonic acid, calcium salt of 2-hydroxy-1,2′-azonaphthalene-1′-sulphonic acid, calcium and barium salts of 1-(2-sulpho-4-methyl-1-phenylazo)-2-naphthylcarboxylic acid, calcium salt of 1-(2-sulpho-1-naphthylazo)-2-hydroxynaphthalene-3-carboxylic acid, aluminium salt of 1-(4-sulpho-1-phenylazo)-2-naphthol-6-sulphonic acid, aluminium salt of 1-(4-sulpho-1-naphthylazo)-2-naphthol-3,6-disulphonic acid, 1-(4-sulpho-1-naphthylazo)-2-naphthol-6,8-disulphonic acid, aluminium salt of 8-amino-2-phenylazo-1-naphthol-3,6-disulphonic acid, aluminium salt of 4-(4-sulpho-1-phenylazo)-1-(4-sulphophenyl)-5-hydroxypyrazolone-3-carboxylic acid, 4′-[(4″-sulpho-1″-phenylazo)-7′-sulpho-1′-naphthylazo]-1-hydroxy-8-acetylaminonaphthalene-3,5-disulphonic acid, aluminium and zirconium salts of 4,5-dibromofluorescein, aluminium and zirconium salts of 2,4,5,7-tetrabromofluorescein, 3′,4′,5′,6′-tetrachloro-2,4,5,7-tetrabromofluorescein and its aluminium salt, aluminium salt of 2,4,5,7-tetraiodofluorescein, aluminium salt of quinophthalonedisulphonic acid, aluminium salt of indigodisulphonic acid, 4,4′-dimethyl-6,6′-dichlorothioindigo, complex salt (Na, Al, Ca) of carminic acid, red and black iron oxide (CIN: 77 491 (red) and 77 499 (black)), iron oxide hydrate (CIN: 77 492), manganese ammonium diphosphate (CIN 77745), ultramarine (CIN 77007) and titanium dioxide.

According to the invention, apart from the abovementioned substances, the compositions optionally comprise the additives customary in cosmetics, for example dyes, antimicrobial substances, refatting agents, complexing and sequestering agents, pearlizing agents, plant extracts, vitamins, active ingredients, antidandruff active ingredients, preservatives, bactericides, pigments which have a colouring effect, thickeners, softening, moisturizing and/or humectant substances, or other customary constituents of a cosmetic or dermatological formulation, such as alcohols, polyols, polymers, UV photoprotective filters, foam stabilizers, electrolytes, organic solvents or silicone derivatives.

According to the invention, cosmetic cleansing gels can comprise cosmetic auxiliaries as are customarily used in such preparations, e.g. preservative aids, complexing agents, antioxidants, buffers, solubility promoters, dispersants, bactericides, perfumes, further substances for preventing or increasing foaming, dyes, pigments which have a colouring effect, thickeners, pearlescent pigments, softening, moisturizing and/or humectant substances (e.g. urea), fats, oils, waxes or other customary constituents of a cosmetic or dermatological formulation, such as alcohols, polyols, polymers, omega-fatty acids, foam stabilizers, electrolytes, organic solvents or silicone derivatives.

In particular, it is advantageous according to the invention to add vitamins and plant extracts to the preparation according to the invention. Thus, for example, the addition of calcium vitamin complexes, niacinamide and/or panthenol is particularly advantageous according to the invention.

A further active ingredient advantageous according to the invention is, for example, polidocanol.

It is also within the scope of the present invention to add to the preparation according to the invention pearlescent pigments, mica, glitter substances, beads and/or effect pigments, peeling particles, such as, for example, polyethylene, in order to make the preparation more visually attractive and/or to bring about an additional use. A preparation according to the invention in the form of a gel with gas bubbles, in particular air bubbles, or streaks, in particular coloured streaks, can also be provided in a manner advantageous for the invention.

The preparation can additionally comprise abrasives, e.g. polymer beads or powders made of polyethylene, polypropylene or inorganic oxides or silicates. According to the invention, these have an average particle size of less than 400 μm, preferably less than 300 μm, particularly preferably between 250 to 75 μm, in an amount of from 0.1 to 3% by weight, based on the total weight of the formulation, individually or as a mixture of two or more abrasives.

According to the invention, the preparation according to the invention or in the case of the preparation in which the use according to the invention is realized advantageously has a pH of from pH 4 to pH 7.

According to the invention, the preparation according to the invention or the preparation in which the use according to the invention is realized is a cleansing gel. Here, transparent cleansing gels are preferred according to the invention.

According to the invention, the preparation according to the invention or the preparation in which the use according to the invention is realized is advantageously a cleansing gel for cleansing the skin of face and body. According to the invention, particular preference here is given to gels for cleansing facial skin (either dry skin, normal combination skin or else greasy-oily skin with a tendency towards acne). However, the present invention can also advantageously be realized according to the invention in hair shampoos.

It is advantageous for the purposes of the present invention if the cosmetic cleansing gel according to the invention ascertains a viscosity of from 4000 to 8000 mPas (Viskotester VT 02 from Haake, temperature: 25° C., spindle: rotating body 1 (24 mm diameter), rotary speed: 62.5 min⁻¹).

The packaging according to the invention can be a packaging container made of glass or plastic. Preference is given according to the invention here to plastic containers. (PE, PP, PET). It is also advantageous according to the invention if the packaging container is not coloured, but colourless. According to the invention preference is given to transparent packagings.

The examples below are intended to explain the compositions according to the invention, but are not intended to limit the invention to these examples. The numerical values in the examples are percentages by weight, based on the total weight of the particular preparations.

Shower gels:

| | 1 | 2 | 3 | 4 | 5 |
|---|---|---|---|---|---|
| Sodium laureth sulphate | 13.2% | 11% | 9.5% | 11% | 9.5% |
| Cocoamidopropylbetaine | 1.65% | 3.3% | 3.8% | 3.3% | 3.8% |
| Sodium cocoyl glutamate | 1.25% | 0.75% | 2.5% | 0.75% | 0.75% |
| Hydroxypropyldistarch phosphate | 0.2% | 0.5% | 1% | 0.2% | 1% |
| (Structure ® XL) | | | | | |
| PEG-7 glyceryl cocoate | — | — | — | 2.0% | 2.0% |
| PEG-40 hydrogenated castor oil | 0.50% | 0.50% | 0.5% | 0.50% | 0.50% |
| Ubiquinone | — | 0.01% | — | — | 0.025% |
| Licochalcone A | 0.025% | — | 0.05% | 0.02% | 0.02% |
| Polyquaternium-10 | 0.2% | — | 0.2% | — | — |
| Sodium benzoate | 0.45% | 0.45% | 0.45% | 0.45% | 0.45% |
| Sodium salicylate | 0.20% | 0.20% | 0.2% | 0.20% | 0.20% |
| Citric acid | 0.50% | 0.50% | 0.5% | 0.50% | 0.50% |
| Perfume | q.s. | q.s. | q.s. | q.s. | q.s. |
| Water | ad 100 | ad 100 | ad 100 | ad 100 | ad 100 |

LES-free shower gels

| | 1 | 2 | 3 |
|---|---|---|---|
| Sodium myreth sulphate | 5% | 4% | 6% |
| Lauryl glucoside | 2.5% | — | — |
| Decyl glucoside | — | 3% | — |
| Sodium cocoamphoacetate | 6.5% | 7% | 8% |
| Hydroxypropyldistarch phosphate | 0.5% | 0.2% | 1% |
| (Structure ® XL) | | | |
| PEG-200 hydrogenated glyceryl | 0.4% | 0.4% | 0.4% |
| palmitate | | | |
| PEG-40 hydrogenated castor oil | 1% | 1% | 1% |
| Diammonium citrate | 0.12% | 0.12% | 0.12% |
| Polyquaternium-10 | 0.2% | — | — |
| Licochalcone A | 0.025% | — | 0.05% |
| Ubiquinone | 0.01% | 0.025% | — |
| Sodium benzoate | 0.3% | 0.3% | 0.3% |
| Sodium salicylate | 0.2% | 0.2% | 0.2% |
| Citric acid | 1.2% | 1.2% | 1.2% |
| Perfume | q.s. | q.s. | q.s. |
| Water | ad 100 | ad 100 | ad 100 |

Face cleansing gels

| | 1 | 2 | 3 | 4 | 5 |
|---|---|---|---|---|---|
| Sodium myreth sulphate | 2% | 4% | 3% | 5% | 2% |
| Decyl glucoside | 2% | 2% | 4% | 1% | 4% |
| Cocoamidopropylbetaine | — | 2% | — | 1% | 1% |
| Carbopol 1382 | 0.3% | 0.6% | 0.5% | 1% | — |
| Acrylates copolymer | 0.3% | 0.5% | 0.2% | 0.2% | 1% |
| Hydroxypropyldistarch phosphate | 0.2% | 0.5% | 1% | 0.2% | 1% |
| (Structure ® XL) | | | | | |
| Sodium hydroxide | 0.5% | 0.5% | 0.5% | 0.5% | 0.5% |
| Glycerol | 5% | 10% | 5% | 10% | — |
| Na₃HEDTA | 0.5% | 0.5% | 0.5% | 0.5% | 0.5% |
| Polyethylene | — | 0.1% | 0.4% | — | — |
| Licochalcone A | 0.025% | — | 0.05% | 0.02% | 0.02% |
| Ubiquinone | — | 0.01% | — | — | 0.025% |
| Phenoxyethanol | 0.5% | 0.5% | 0.5% | 0.5% | 0.5% |
| Parabens | 0.2% | 0.2% | 0.2% | 0.2% | 0.2% |
| Perfume | q.s. | q.s. | q.s. | q.s. | q.s. |
| Water | ad 100 | ad 100 | ad 100 | ad 100 | ad 100 |
| | 6 | 7 | 8 | 9 | 10 |
| Sodium laureth sulphate | 2% | 2% | 7% | 7% | — |
| Methyl cocoyltaurate | 0.6% | 0.6% | 0.6% | 0.6% | 6% |
| Carbopol 980 | 1.2% | 1.2% | 1.2% | 0.5% | 0.6% |
| Hydroxypropyldistarch phosphate | 0.2% | 0.5% | 1% | 0.2% | 1% |
| (Structure ® XL) | | | | | |
| Sodium hydroxide | 0.5% | 0.5% | 0.5% | 0.5% | 0.5% |
| Glycerol | 2.0% | 2.0% | 2.0% | — | 2.0% |
| Polyethylene | 0.1% | 0.3% | 0.4% | — | — |
| Licochalcone A | 0.025% | — | 0.05% | 0.02% | 0.02% |
| Ubiquinone | — | 0.01% | — | — | 0.025% |
| Xanthan gum | 0.25% | 0.1% | 0.25% | — | — |
| Phenoxyethanol | 0.5% | 0.5% | 0.5% | 0.5% | 0.5% |
| Parabens | 0.2% | 0.2% | 0.2% | 0.2% | 0.2% |
| Perfume | q.s. | q.s. | q.s. | q.s. | q.s. |
| Water | ad 100 | ad 100 | ad 100 | ad 100 | ad 100 |

1. A cosmetic preparation comprising one or more labile ingredients, and one or more pregelatinized, crosslinked starch derivatives.

2. The cosmetic preparation according to claim 1, wherein the one or more pregelatinized, crosslinked starch derivatives include a hydroxypropylated phosphate ester of starch.

3. The cosmetic preparation according to claim 1, wherein the one or more pregelatinized, crosslinked starch derivatives include a hydroxypropyidistarch phosphate.

4. The cosmetic preparation according to claim 1, wherein the one or more pregelatinized, crosslinked starch derivatives are present in an amount of 0.05 to 5% by weight of the preparation.

5. The cosmetic preparation according to claim 1, wherein the one or more labile ingredients include one or more labile ingredients selected from the group consisting of licochalcone A, ubiquinone Q-10, and combinations thereof.

6. The cosmetic preparation according to claim 5, wherein the total amount of licochalcone A and ubiquinone Q-10 in the preparation is 0.001 to 0.3% by weight of the preparation.

7. The cosmetic preparation according to claim 1, wherein the one or more labile ingredients include a perfume substance.

8. The cosmetic preparation according claim 7, wherein the total amount of perfume substance in the preparation is from 0 to 2% by weight, based on the total weight of the preparation.

9. The cosmetic preparation according to claim 1, wherein the cosmetic preparation is a cleansing gel.

10. The cosmetic preparation according to claim 1, further comprising a surfactant selected from the group consisting of anionic surfactants, cationic surfactants, amphoteric surfactants, non-ionic surfactants, and combinations thereof.

11. A cosmetic product, comprising:
a transparent or translucent package containing a cosmetic preparation;
wherein the cosmetic preparation comprises:
one or more labile ingredients, and
one or more pregelatinized, crosslinked starch derivatives.

12. The cosmetic product of claim 11, wherein the one or more pregelatinized, crosslinked starch derivatives include a hydroxypropylated phosphate ester of starch.

13. The cosmetic product of claim 11, wherein the one or more pregelatinized, crosslinked starch derivatives include a hydroxypropyldistarch phosphate.

14. The cosmetic product of claim 11, wherein the one or more pregelatinized, crosslinked starch derivatives are present in an amount of 0.05 to 5% by weight of the preparation.

15. The cosmetic product of claim 11, wherein the one or more labile ingredients include one or more labile ingredients selected from the group consisting of licochalcone A, ubiquinone Q-10, and combinations thereof.

16. A method of stabilizing one or more labile ingredients in a cosmetic preparation, comprising the step of combining one or more labile ingredients with one or more pregelatinized, crosslinked starch derivatives.

17. The method of claim 16, wherein the step of combining comprises combining the one or more labile ingredients with a hydroxypropylated phosphate ester of starch.

18. The method of claim 16, wherein the step of combining comprises combining the one or more labile ingredients with a hydroxypropyidistarch phosphate.

19. The method of claim 16, wherein the step of combining comprises combining the one or more pregelatinized, crosslinked starch derivatives in an amount of 0.05 to 5% by weight of the preparation.

20. The method of claim 16, wherein the step of combining comprises combining the one or more pregelatinized, crosslinked starch derivatives with one or more labile ingredients selected from the group consisting of licochalcone A, ubiquinone Q-10, and combinations thereof.
